(19) United States
(12) Patent Application Publication
(10) Pub. No.: US 2006/0263834 A1
(43) Pub. Date: Nov. 23, 2006

(54) Functional multi-drug resistance assay

(75) Inventor: Michael R. Loken, Mercer Island, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: HematoLogics, Inc., Seattle, WA (US)

(21) Appl. No.: 11/361,695

(22) Filed: Feb. 24, 2006

Related U.S. Application Data
(60) Provisional application No. 60/656,336, filed on Feb. 25, 2005.

(51) Int. Cl. G01N 33/574; C07K 16/30; C07K 16/46

(52) U.S. Cl. 435/7.23; 530/391.1

(57) ABSTRACT

The invention provides methods for determining multi-drug resistance activity of a tumor cell that is present in a heterogeneous mixture of normal cells. The present invention relates particularly to improved quantitative methods for characterizing multi-drug resistance of selected populations of cells, including hematopoietic cell populations containing malignant cells. More specifically, the invention provides flow immunocytometric methods for determining whether malignant hematopoietic cells are resistant to a chemotherapeutic drug and for identifying agents that inhibit multi-drug resistance activity in the malignant cells.

### BACKGROUND OF THE INVENTION

1. Technical Field

The present invention relates generally to improved quantitative methods for characterizing multi-drug resistance of selected populations of cells, including hematopoietic cell populations containing malignant cells. More specifically, the invention provides a flow immunocytometric method for determining whether malignant hematopoietic cells may be resistant to a chemotherapeutic drug and for identifying agents that inhibit multi-drug resistance in the cells.

2. Description of the Related Art

Cancer represents a broad constellation of diseases typically characterized by rapid and unregulated proliferation of malignant cells. Because approximately 25% of the human population is affected by one form or another, cancer poses a major world health problem. Neoplastically transformed cells arising from hematopoietic cells, for example, malignancies such as acute or chronic leukemias, acute or chronic lymphomas, myelodysplasias and the like, are particularly problematic, given that steady-state production and turnover of hematopoietic cells is required for survival and that cells of hematopoietic origin circulate throughout the body.

Various chemotherapeutic agents have been used to treat patients who have been diagnosed with a hematopoietic malignancy. Tumor cells are approximately five-fold more sensitive to anti-cancer drugs than are healthy cells. This narrow therapeutic window permits the use of cytotoxic agents to destroy malignancies. During chemotherapy, however, tumor cells often become less sensitive to these agents and become as refractory as normal cells. This diminished sensitivity to a single agent often extends to a broad class of other drugs, which are diverse in their structures and targets. The acquired or inherent multi-drug resistance (MDR) is a major challenge to successful chemotherapy of malignant tumors.

Because resistance of malignant tumors to chemotherapeutic agents remains a major cause of failure in cancer therapy, a need exists for methods that determine the level of resistance of a tumor cell to a chemotherapeutic drug and that monitor development of drug resistance throughout a course of therapy. The present invention provides methods that satisfy these needs and offers other related advantages.

### BRIEF SUMMARY OF THE INVENTION

The present invention relates generally to improved methods for determining whether a tumor cell has multi-drug resistance activity and for identifying agents that inhibit multi-drug resistance activity in a tumor cell.

One aspect of the present invention provides a method for identifying an agent that inhibits multi-drug resistance activity in a tumor cell comprising; contacting (i) a biological sample comprising a plurality of cells, (ii) a fluorescent dye that is capable of being transported across a cell membrane, and (iii) at least a first antibody and a second antibody, wherein the first antibody specifically binds to a first cell surface antigen that is present on a normal cell and the second antibody specifically binds to a second cell surface antigen that is present on a tumor cell, and wherein the first antibody is detectably labeled with a first fluorophore and the second antibody is detectably labeled with a second fluorophore, wherein each fluorophore has a distinguishable emission spectra from each other and the dye, under conditions and for a time sufficient to permit interaction among the plurality of cells, the dye, and the antibodies; isolating the plurality of cells from the biological sample; retaining a first aliquot of the plurality of cells under conditions that inhibit efflux of the dye from a cell; retaining a second aliquot of the plurality of cells under conditions and for a time sufficient to permit efflux of the dye from a cell; retaining a third aliquot of the plurality of cells and contacting the plurality of cells with a candidate agent under conditions and for a time that permit interaction between the cells and the agent; detecting the level of fluorescence of the dye in cells to which the first antibody binds and in cells to which the second antibody, in each of the three aliquots of cells; comparing in the first, second, and third aliquots the level of fluorescence of the dye in the plurality of cells to which the first antibody binds, wherein a decreased level of fluorescence of the dye in the second aliquot compared with the level of fluorescence of the dye in the first aliquot indicates efflux of the dye from the normal cell, and wherein an increase in the level of fluorescence of the dye in the third aliquot compared with the level of fluorescence of the dye in the second aliquot indicates that the agent inhibits efflux of the dye from the cell, thereby indicating that the normal cell has multi-drug resistance activity and thereby providing an internal control for determining multi-drug resistance activity; and comparing in the first, second, and third aliquots the level of fluorescence of the dye in the plurality of cells to which the second antibody binds, wherein a decreased level of fluorescence of the dye in the second aliquot compared with the level of fluorescence of the dye in the first aliquot indicates efflux of the dye from the tumor cell, and wherein an increase in the level of fluorescence of the dye in the third aliquot compared with the level of fluorescence of the dye in the second aliquot indicates that the agent inhibits efflux of the dye from the tumor cell, thereby identifying an agent that inhibits multi-drug resistance activity in a tumor cell.

An additional aspect of the present invention provides a method for detecting multi-drug resistance activity of a tumor cell comprising: contacting (i) a biological sample comprising a plurality of cells, (ii) a fluorescent dye that is capable of being transported across a cell membrane, and (iii) at least a first antibody and a second antibody, wherein the first antibody specifically binds to a first cell surface antigen that is present on a normal cell and the second antibody specifically binds to a second cell surface antigen that is present on a tumor cell, and wherein the first antibody is detectably labeled with a first fluorophore and the second antibody is detectably labeled with a second fluorophore, wherein each fluorophore has a distinguishable emission spectra from each other and the dye, under conditions and for a time sufficient to permit interaction among the plurality of cells, the dye, and the antibodies; isolating the plurality of cells from the biological sample; retaining a first aliquot of the plurality of cells under conditions that inhibit efflux of the dye from a cell; retaining a second aliquot of the plurality of cells under conditions and for a time sufficient that permit efflux of the dye from a cell; contacting a third aliquot of the plurality of cells with an agent that inhibits multi-drug resistance activity, under conditions and for a time that permit interaction between the cells and the agent; detecting the level of fluorescence of the dye in cells to which the first antibody binds and in cells to which the second antibody, in each of the three aliquots of cells; comparing in the first, second, and third aliquots the level of fluorescence of the dye in the plurality of cells to which the first antibody binds, wherein a decreased level of dye in the second aliquot compared with the level of fluorescence of the dye in the first aliquot indicates efflux of the dye from the normal cell, and wherein an increase in the level of fluorescence of the dye in the third aliquot compared with the level of fluorescence of the dye in the second aliquot indicates that the agent inhibits efflux of the dye from the normal cell, thereby indicating that the normal cell has multi-drug resistance activity and thereby providing an internal control for determining multi-drug resistance activity; and comparing in the first, second, and third aliquots the level of fluorescence of the dye in the plurality of cells to which the second antibody binds, wherein a decreased level of fluorescence of the dye in the second aliquot compared with the level of fluorescence of the dye in the first aliquot indicates efflux of the dye from the tumor cell, and wherein an increase in the level of fluorescence of the dye in the third aliquot compared with the level of fluorescence of the dye in the second aliquot indicates that the agent inhibits efflux of the dye from the tumor cell, thereby indicating that the tumor cell has multi-drug resistance activity inhibitable by the agent.

A further aspect of the present invention provides a method for monitoring drug resistance activity of a tumor cell from a subject who has a malignant condition, said method comprising: obtaining a biological sample from a subject who has a malignant condition; contacting (i) a biological sample comprising a plurality of cells, (ii) a fluorescent dye that is capable of being transported across a cell membrane, and (iii) at least a first antibody and a second antibody, wherein the first antibody specifically binds to a first cell surface antigen that is present on a normal cell and the second antibody specifically binds to a second cell surface antigen that is present on a tumor cell, and wherein the first antibody is detectably labeled with a first fluorophore and the second antibody is detectably labeled with a second fluorophore, wherein each fluorophore has a distinguishable emission spectra from each other and the dye, under conditions and for a time sufficient to permit interaction among the plurality of cells, the dye, and the antibodies; isolating the plurality of cells from the biological sample; retaining a first aliquot of the plurality of cells under conditions that inhibit efflux of the dye from a cell; retaining a second aliquot of the plurality of cells under conditions and for a time sufficient to permit efflux of the dye from a cell; contacting a third aliquot of the plurality of cells with an agent that inhibits multi-drug resistance activity under conditions and for a time that permit interaction between the cells and the agent; detecting the level of fluorescence of the dye in cells to which the first antibody binds and in cells to which the second antibody, in each of the three aliquots of cells; comparing in the first, second, and third aliquots the level of fluorescence of the dye in the plurality of cells to which the first antibody binds, wherein a decreased level of fluorescence of the dye in the second aliquot compared with the level of fluorescence of the dye in the first aliquot indicates efflux of the dye from the normal cell, and wherein an increase in the level of fluorescence of the dye in the third aliquot compared with the level of fluorescence of the dye in the second aliquot indicates that the agent inhibits efflux of the dye from the cell, thereby indicating that the normal cell has multi-drug resistance activity and thereby providing an internal control for determining multi-drug resistance activity; and comparing in the first, second, and third aliquots the level of fluorescence of the dye in the plurality of cells to which the second antibody binds, wherein a decreased level of fluorescence of the dye in the second aliquot compared with the level of fluorescence of the dye in the first aliquot indicates efflux of the dye from the tumor cell, and wherein an increase in the level of fluorescence of the dye in the third aliquot compared with the level of fluorescence of the dye in the second aliquot indicates that the agent inhibits efflux of the dye from the tumor cell, thereby indicating that the tumor cell has multi-drug resistance activity.

In one embodiment of the methods provided herein, the biological sample can be from any one or more of blood, bone marrow, lymph node, cerebrospinal fluid, ascites fluid, pleural fluid, pericardial fluid, peritoneal fluid, and lavage fluid. In one particular embodiment, the biological sample is obtained from a subject who has a malignant condition. In a further embodiment, the plurality of cells comprise a heterogeneous mixture of cell types. In yet a further embodiment of the methods described herein, the normal cell is a natural killer cell. In this regard, in certain embodiments, the first cell surface antigen is CD56 or CD11 and the normal cell is a natural killer cell. In yet a further embodiment, the first cell surface antigen is either CD56, CD11 or CD16. In an additional embodiment, the second cell surface antigen can be any one or more of CD45, CD34, CD33, CD13, and CD38. In one embodiment, the tumor cell is a leukemic blast cell. In another embodiment, the second cell surface antigen is CD45 and the tumor cell is a leukemic blast cell. In yet a further embodiment, the second cell surface antigen is CD34 and the tumor cell is a leukemic blast cell.

In an additional embodiment, the methods described herein further comprises contacting the biological sample and the dye with a third antibody that specifically binds to a third cell surface antigen that is present on a tumor cell, and wherein the third antibody is detectably labeled with a third fluorophore that has an emission spectra distinguishable from the emission spectra of the first and second fluorophores and the dye. In one embodiment, the second antibody specifically binds to cell surface antigen CD45 and the third antibody binds to cell surface antigen CD34, and wherein the tumor cell is a leukemic blast cell.

In another embodiment, the method is a cytofluorimetric method, such as a flow cytofluorimetric method. In a further embodiment, the cytofluorimetric method is an immunocytofluorimetric method. In another embodiment, the malignant condition is a leukemia. In yet a further embodiment of the methods described herein, illustrative dyes include, but are not limited to DuIC, DiOC, Rhodamine 123, and JC-1.

In certain embodiments of the methods described herein, the first antibody is directly or indirectly labeled with the first fluorophore. In certain additional embodiments, the second antibody is directly or indirectly labeled with the second fluorophore. In a further embodiment, the third antibody is directly or indirectly labeled with the third fluorophore. In yet a further embodiment, the first antibody is a monoclonal antibody, or an antigen-binding fragment thereof. In an additional embodiment, the second antibody is a monoclonal antibody, or an antigen-binding fragment thereof. In yet another embodiment, the third antibody is a monoclonal antibody, or an antigen-binding fragment thereof.

In certain embodiments of the methods described herein, the tumor cell is a leukemia cell or a lymphoma cell. In another embodiment, the leukemia cell may be an acute myelogenous leukemia cell, a chronic myelogenous leukemia cell, an acute lymphocytic leukemia cell, or a chronic lymphocytic leukemia cell. In an additional embodiment, the lymphoma cell may be a Hodgkin's lymphoma cell, a non-Hodgkin's lymphoma cell, a T lymphoblastoid lymphoma cell, or a B lymphoblastoid lymphoma cell. In a further embodiment, detection of any one of (a) the first antibody specifically binding to the first cell surface antigen and (b) the second antibody specifically binding to the second cell surface antigen, comprises detection of a binding event between an avidin molecule and a biotin molecule. In another embodiment, detection of the third antibody specifically binding to the third cell surface antigen comprises detection of a binding event between an avidin molecule and a biotin molecule.

These and other aspects of the present invention will become evident upon reference to the following detailed description. In addition, various references are set forth herein that describe in more detail certain aspects of this invention. All cited references, patents, and patent applications including those listed on the application data sheet (ADS) are incorporated herein in their entirety as if each was incorporated individually.

### BRIEF DESCRIPTION OF THE DRAWINGS

FIGS. 1A and 1B: Flow cytometric analysis of bone marrow cells from a patient with acute myeloid leukemia: FIG. 1A: internal control NK cells; Panel A: aliquot incubated at 4 degrees C. (equivalent to group 1N; see Example 1 and 2). Panel B: aliquot incubated at 37 degrees C. (equivalent to group 2N; see Example 1 and 2). Panel C: aliquot incubated at 37 degrees C. in the presence of MDR-1 inhibitor (equivalent to group 3N; see Example 1 and 2). FIG. 1B: leukemic blast cells: Panel A: aliquot incubated at 4 degrees C. (equivalent to group 1C; see Example 1 and 2). Panel B: aliquot incubated at 37 degrees C. (equivalent to group 2C; see Example 1 and 2). Panel C: aliquot incubated at 37 degrees C. in the presence of MDR-1 inhibitor (equivalent to group 3C; see Example 1 and 2).

### DETAILED DESCRIPTION OF THE INVENTION

The present invention relates to methods for identifying patients, who have a malignant condition, for example, a hematopoietic malignancy, for their suitability to receive a chemotherapeutic agent for treating the malignancy. The invention pertains in part to an improved method for determining whether a tumor cell (malignant cell or cancer cell) has multi-drug resistance (MDR) activity or is developing MDR activity, and for determining the level of such activity. The presence of MDR in a tumor cell from a patient can be determined prior to initial administration of a chemotherapeutic drug to the patient and/or during the course of a chemotherapy regimen to determine whether a tumor cell is developing MDR. Such methods are useful to a person skilled in the medical art to determine a suitable therapeutic regimen and to alter the regimen if MDR activity is increasing in the tumor cells. In another embodiment, the invention provides a method for identifying agents that may be useful for inhibiting (decreasing, abrogating, or minimizing in a statistically significant manner or clinically significant manner) MDR activity in the tumor cell.

The assessment of MDR activity requires the cells first to be labeled with a dye then incubated under conditions whereby the dye is pumped out of the cell. This is a functional assay and is difficult to properly control under routine conditions in a clinical laboratory since the specimen is heterogeneous, the cells of interest may be frequent or infrequent and the integrity of the specimen may be compromised in collection, shipping or storage. Therefore a test for MDR activity should identify the tumor cells of interest and an internal normal cell control that demonstrates MDR function should be assayed along with the tumor cells. In addition, only a subset of tumor may have this function so identifying the tumor stem cell may be important in identifying MDR activity in a subset of tumor.

Furthermore, the functional assays are generally conducted at 37 degrees C. under conditions where the cells can metabolize. These are conditions where the antigens and antibodies selected for identifying appropriate cells can be shed from the cells and are lost. This requires careful selection of appropriate cell surface antigens that are maintained during the incubation periods.

The present invention provides a functional assay that reduces variability by labeling the cells for cell surface antigens and the MDR dye simultaneously in a sufficient quantity so a large aliquot of cells is labeled for subsequent assessment of function. Therefore, a single group of cells is labeled for cell surface antigens and MDR dye. Once adequately labeled, the cell suspension is divided into two or more groups for further incubation under conditions which promote MDR function and conditions which inhibit MDR function. As described further herein, such conditions include incubation in the cold (inhibiting MDR function), warm (37 degrees C. permitting MDR function) or warm with drugs that inhibit MDR function. Cell populations can then be analyzed immediately after the incubation times to reduce potential leakage of the MDR dye through further processing.

Selection of cell surface markers that identify the correct cell populations is crucial in this assay. These markers must survive the incubation procedures so they can be used to identify the various cell populations to assess their MDR function.

Typically cells are labeled with an MDR dye at 37 degrees C. The cells take up the dye and fluoresce. MDR function can be inhibited by cooling the cells to 4 degrees C. and the intensity of the dye remains constant for an hour or more. Cells with MDR pump out the dye at 37 degrees therefore comparing the fluorescence intensity of two cell populations permits the assessment of the MDR pump. Cells maintained in the cold will have greater fluorescence than cells incubated at 37 degrees. The difference is a measure of the function of the MDR pump. This process is simple when the cells to be tested are homogeneous, i.e., a cell line. However, tumor cells generally are mixed with other normal cells. Accordingly, the tumor cells must be somehow distinguished from the normal cells. In addition, the tumor cells may be heterogeneous with regards to MDR function, with some tumor cells having an active MDR pump while others lack the MDR pump. Further, it is important to subdivide the tumors into the fraction that is most likely to include the tumor stem cell.

Normal cells in blood or bone marrow that demonstrate MDR include NK (natural killer cells). Leukemia blasts can be identified by CD45 and right angle light scatter. These leukemic blasts can be further subdivided into immature vs mature using the antigen CD34. Therefore, the assay of the present invention identifies NK cells, all leukemia cells and subsets of leukemia cells and then assesses each of these populations for MDR function.

As described further herein, NK cells can be detected by CD56, CD16 or CD11b. Of the known NK cell markers, only CD11b survives incubation at 37 degrees for 30 minutes. Accordingly, the present invention employs CD11b for the identification of NK cells. As would be understood by the skilled artisan, other markers could be used as long as they survive the incubation. For example, CD56 or CD16 may be useful with some stabilization measures. In a similar manner, total leukemia can be distinguished from normal cells by CD45 and right angle light scatter. CD45 survives incubation at 37 degrees for 2 or more hours. In addition, CD34, an antigen expressed on immature hematopoietic progenitor cells and on subsets of leukemic cells (including presumably the most immature leukemia stem cell) also remains on the cell surface following incubation at 37 degrees C.

By labeling the bone marrow cells with antibodies specific for CD11b, CD45, CD34 and the MDR dye simultaneously, it is possible to identify all cell populations of interest in a bone marrow or blood specimen and compare the intensity of the MDR dye for each of the cell populations in a sample. In one embodiment, the bone marrow cells are labeled with all the dyes for about 30 minutes at 37 degrees, the cells are washed then divided into aliquots for incubation at 4 degrees, 37 degrees, and 37 degrees with an MDR inhibitor for 30 minutes to 1 hour. The fluorescence of the MDR dye for each of these aliquots is then assessed with minimal further handling thereby minimizing the variability between aliquots.

Multi-Drug Resistance

A multigene family has been identified that encodes a family of membrane glycoproteins that are recognized for mediating multi-drug resistance. These glycoproteins are members of the ATP binding cassette (ABC) transporter family. ABC transporters transport substrates against a concentration gradient with ATP hydrolysis as a driving force across the membrane. Mammalian ABC proteins have important physiological, pharmacological, and toxicological functions including the transport of lipids, bile salts, drugs, toxic and environmental agents. The efflux pumps serve both as natural defense mechanisms and influence the bioavailability and disposition of drugs. ABC transporters that are recognized as multidrug resistance proteins include MDR1 (P-glycoprotein, ABCB1), MRP2 (ABCC2), and Bcrp1/ABCG2 (see, e.g., Endicott et al., Ann. Rev. Biochemi. 58:137-71 (1989); Chen et al., Cell 47:381-89 (1986); Hoffman et al., Drug Metab. Rev. 36:669-701 (2004); Israeli et al., J. Theor. Biol. 232:41-45 (2005); U.S. Pat. Nos. 5,851,819; 5,206,352, 4,912,039).

MDR proteins act as ATP-dependent efflux pumps capable of effluxing a variety of cytotoxic agents, decreasing their intracellular concentration. A MDR protein may also act as a flippase, which is an art-recognized term that is intended to include the ability of an ABC transporter to act as a flipping agent, that is, moving a molecule or compound from the inner leaflet of a lipid bilayer to the outer leaflet. Alternatively, an MDR protein may move or flip a molecule from the outer leaflet of the lipid bilayer of a cell into the extracellular space.

One or more MDR proteins are overexpressed in multidrug resistant cell lines, which display cross-resistance to a broad spectrum of structurally and functionally unrelated compounds. Compounds or drugs that are transported out of a cell that exhibits the multi-drug resistance phenotype include many of the most potent natural product agents currently used in cancer chemotherapy, for example, the antracyclines, Vinca alkaloids, epidophyllotoxins, and certain protein synthesis inhibitors such as actinomycin D.

Methods for Determining MDR

Assays that are currently used to determine MDR activity in malignant cells were developed using cell lines, which comprise a homogeneous population of cells. These assays when used to assess MDR in hematopoietic malignancies, such as leukemia, generally are based upon the assumption that the malignant cells comprise the majority of the cell population. For patients with leukemia, the biological sample that is evaluated in the assay is most often bone marrow, which is heterogeneous; that is, the bone marrow comprises multiple lineages of cells that are at different stages of maturation. Such biological samples that contain heterogeneous cell populations therefore comprise at least two different cell types, and generally comprise more than two different cell types. Therefore, the leukemic sample is not like a cell line but instead the malignant cells are mixed with a variety of other cells. Normal cells also express MDR proteins; therefore, distinguishing the malignant cells from the normal cells to identify specifically the presence of MDR proteins in the malignant cells and to determine the function of MDR proteins in the malignant cells is most useful. In addition, most assays presently in use require laborious multi-step purification of cells using ficoll and/or red cell lysing techniques to separate and remove red blood cells from the biological sample. Methods described herein are improved, less laborious methods that are useful for determining MDR activity in normal cells and in tumor (malignant) cells using biological samples that contain heterogeneous cell populations and that incorporate internal positive and negative controls.

Many compounds have been identified that are capable of inhibiting efflux of a chemotherapeutic agent or other compound from a cell by inhibiting MDR activity in a multi-drug resistant cell. Compounds that interact with an MDR protein, such as p-glycoprotein, and inhibit its efflux function include, but are not limited to, verapamil, quinine, progesterone, tamoxifen, atropine, forskolin, and cyclosporin (see, e.g., U.S. Pat. No. 6,660,725 and references therein for a description of compounds that block transporter or flippase activity). However, many of these compounds can have toxic effects on normal cells. Accordingly, in one embodiment of the invention, a screening method is provided for identifying an agent that inhibits MDR activity (inhibits, decreases, abrogates in a statistically significant or clinically significant manner) in a malignant cell and that determines whether the agent inhibits transporter function in a normal cell.

The methods described herein that determine MDR activity are useful for (a) determining whether a subject who has a hematopoietic malignancy is likely to respond to treatment with a therapeutic agent, such as a chemotherapeutic agent; (b) determining whether a tumor cell, which may be isolated from a biological sample obtained from a subject, has multi-drug resistance activity; (c) monitoring the effectiveness of a chemotherapeutic agent regimen by determining over the course of chemotherapy whether tumor cells are developing resistance to the chemotherapeutic drugs administered to a subject; (d) identifying an agent or compound that inhibits multi-drug resistance activity in a tumor cell. As described herein, the methods are particularly useful in the context of hematologic disorders such as hematopoietic neoplasias, including malignant conditions such as leukemia and lymphoma.

To determine whether a tumor cell has or is developing MDR to a chemotherapeutic agent, efflux of the agent from the cell may be measured. Alternatively, efflux of a surrogate agent or a suitable alternate agent may be used in a method for determining MDR activity. Accordingly, in certain embodiments, a lipophilic dye (also referred to herein as an MDR dye) may be used to determine MDR activity. Lipophilic dyes, which include but are not limited to DiIC, DiOC, Rhodamine 123, and JC-1 (Molecular Probes, Eugene, Oreg.) are capable of diffusing into cell membranes. The methods described herein to determine MDR activity quantify the amount of dye that is present in a cell and thus can determine efflux of the dye from the cell (or transport of the dye across a cell membrane). Without wishing to be bound by theory, when a cell exhibits MDR activity, for example, when the cell expresses one or more ABC transporters that transport or move a substrate across a membrane, the lipophilic dye is transported across the cell membrane from the cell into the extracellular space.

In one embodiment, the method described herein is a four-color flow cytometry method. Three antibodies, for example, one antibody that specifically binds to a cell surface antigen that is expressed on a normal cell and two antibodies that bind to cell surface antigens on a tumor cell, (i.e., each of the two antibodies specifically binds to a different cell surface antigen expressed on a tumor cell) are each detectably labeled with a different fluorophore. Each fluorophore has a distinguishable emission spectra from the other fluorophores. The lipophilic dye that is transported across a cell membrane and that is used to determine transport or efflux of the dye from the cell exhibiting MDR activity are selected based on the dye's emission spectra such that the number of antibodies that can be used in the assay to distinguish different cell types can be maximized. In one embodiment, a dye that excites in the red and emits in the far red spectra are used. This permits the use of three different fluorophores attached directly or indirectly to three antibodies with different binding specificities without interference from the brightly staining lipophilic MDR dye. An example of such a dye is DiOC (Molecular Probes, Eugene, Oreg.).

Dyes that have different spectral properties from DiOC and that may be used in the methods described herein include DiIC, a dye that emits in the red spectra (Molecular Probes); another dye with similar properties to DiOC is Rhodamine 123. Such dyes have emission spectra that may not be entirely distinguishable from the emission spectra of certain fluorophores that may be used to directly or indirectly label antibodies that specifically bind to cell surface antigens and accordingly, limit the number of antibodies that may be included in the method. Accordingly, in another embodiment, the method described herein is a three-color flow cytometry method. A dye with a broader emission spectrum such as JC-1 (Molecular Probes), which emits in both the green and orange regions of the optical spectrum, may also be used in the methods described herein.

In one embodiment, a method is provided for identifying an agent that inhibits MDR activity in a tumor cell. The agent thus inhibits transport of a chemotherapeutic agent across a cell membrane and inhibits efflux of the chemotherapeutic agent from the tumor cell. Without wishing to be bound by theory, the agent blocks transport or flippase activity of an energy-dependent ABC transporter that provides the MDR phenotype of a cell as described herein. The methods provided herein are also useful for detecting MDR activity in a tumor cell and for monitoring MDR activity in a tumor cell, for example, during the course of a chemotherapeutic regimen.

A biological sample comprising a plurality of cells (which may be a heterogeneous mixture of different cell types) as described herein is obtained and contacted with (combined, mixed, incubated, permitted to interact with) a fluorescent dye that is capable of being transported across a cell membrane, such as one of the lipophilic dyes described herein and used in the art. The sample and fluorescent dye are also contacted with an antibody (first antibody) that binds specifically to a cell surface antigen (cell surface marker) (first cell surface antigen) that is expressed by and present on a normal cell and another antibody (second antibody) that binds specifically to a different cell surface antigen (cell surface marker) (second cell surface antigen) that is expressed by and is present on the surface of a tumor cell. Each of the antibodies is labeled (directly or indirectly as described herein) with a different fluorophore such that each fluorophore and the dye have distinguishable emission spectra. The biological sample comprising the plurality of cells, fluorescent dye, and antibodies are permitted to interact under conditions and for a time sufficient that permit, for example, the dye to diffuse through a cell membrane and each antibody to bind to its respective cognate cell surface antigen, which conditions and time are described herein and with which a skilled artisan is familiar.

Fluorescent dye that has not diffused into a cell membrane and unwanted cells in the biological sample, for example, red blood cells, may be removed in a single step. For example, mature red blood cells may be lysed and excess dye (not present in a cell) removed by adding ammonium chloride in an appropriate buffer and then isolating the intact cells for example, by centrifugation. To preserve and maintain MDR activity, the biological sample and the cells in the sample and after any isolation step, should be manipulated in a manner that minimizes alterations (decrease) in the ability of the cell to provide energy (that is, ATP) and other components required for transporter function. Such techniques are known and practiced by persons skilled in the art, and include for example, rapid manipulation (handling) of the cells, maintaining proper temperature, and consistent manipulation of cells among different samples.

After the desired population or populations of cells (such as the white blood cells from a bone marrow or blood sample) is isolated (and resuspended in a buffer or media appropriate for maintaining cell viability, which buffers are commonly used and available), the plurality of cells (or suspension of cells) is divided into at least three aliquots. One aliquot or portion is maintained or incubated under conditions that minimize, inhibit, or decrease MDR activity (as well as other metabolic activity of the cells), thus inhibiting efflux of the dye from the cell (inhibiting and/or minimizing transport of the fluorescent dye across a membrane). Such conditions provide a background level of MDR activity and include, for example, maintaining the cells in this aliquot at cold temperatures (such as 0-4° C.). A second aliquot of cells is subjected to conditions that permit MDR activity to occur. Typically, this aliquot of cells is incubated or retained at physiological temperature, 37° C. (for human cells) or other physiological temperature appropriate for the biological source. Alternatively, the cells may be incubated or retained at lower than normal physiological temperatures, such as 30° C. or 25° C. for example, that slow metabolic activity.

To a third aliquot of the plurality of cells, an agent that is known to inhibit MDR activity or is suspected of inhibiting MDR activity is added and incubated under the same conditions as the second aliquot. The level of fluorescence of the dye is then measured in the cells to which the first antibody binds and in the cells to which the second antibody binds, according to methods described herein and practiced in the art, such as fluorescence activated cell sorting (FACS) (flow cytometry). The levels of fluorescence of the dye in the cells to which the first antibody binds and in the cells to which the second antibody binds, are then compared in each aliquot thus comparing normal cells and tumor cells. The level of fluorescence of the dye in the plurality of cells to which the first antibody binds, that is, the normal cells, in the first, second, and third aliquots is compared, which provides an internal control for determining MDR activity. This group of results can be referred to for convenience as 1N (that is, normal cells, bound by the first antibody, first aliquot), 2N (normal cells, bound by the first antibody, second aliquot), and 3N (normal cells, bound by the first antibody, third aliquot). Normal cells that exhibit MDR activity and that are found in biological samples, including bone marrow and blood, are natural killer (NK) cells. A decreased level of dye in the second aliquot (2N) compared with the level of dye in the first aliquot (1N) indicates efflux of the dye from the normal cell, indicating the presence of MDR activity in this population of cells (so, 2N<1N=MDR activity). An increase in the level of fluorescence in the third aliquot (3N) compared with the second aliquot indicates that the agent inhibits efflux of the dye from the cell, thereby indicating that the normal cell has MDR activity and that the agent inhibits MDR activity (so, 3N>2N=inhibited MDR activity). The level of fluorescence of the dye in the third aliquot can also be compared to the level of fluorescence in the first aliquot (the background level) to determine the relative effectiveness of the agent as an inhibitor of MDR activity (e.g., 3N vs 1N).

The level of fluorescence of the dye in the plurality of cells to which the second antibody binds, which are the tumor cells, is compared in each of the three aliquots. This group of results can be referred to for convenience as 1C (that is, cancer cells, bound by the first antibody, first aliquot), 2C (cancer cells, bound by the first antibody, second aliquot), and 3C (cancer cells, bound by the first antibody, third aliquot). Of course, any other convenient designation for these groups can also be used. A decreased level of dye as determined by the fluorescence signal in the second aliquot compared with the level of fluorescence of the dye in the first aliquot indicates efflux of the dye from the tumor cell (2C<1C=MDR activity). An increase in the level of fluorescence of the dye in the third aliquot compared with the second aliquot indicates that the agent inhibits efflux of the dye from the tumor cell (3C>2C=MDR inhibition). Accordingly, such an agent inhibits multi-drug resistance activity in a tumor cell. The level of fluorescence of the dye in the third aliquot can also be compared to the level of fluorescence in the first aliquot (the background level) to determine the relative effectiveness of the agent as an inhibitor of MDR activity in the tumor cell (3C vs 1C).

Other comparisons using the data generated above are also clinically useful. For example, the relative proportion between MDR activity in NK cells and leukemic cells (i.e., 2N vs 2C, 2N/2C) can be used as a means of standardization of the entire system. Also, one can identify cancer cells that are non-responsive to a particular MDR inhibitory agent (i.e., 3C≈2C).

In another embodiment, the method includes contacting or permitting interaction among the biological sample, a first antibody that specifically binds to a first cell surface antigen that is expressed by a normal cell and is present on its cell surface, a second antibody that specifically binds to a cell surface antigen that is expressed by a tumor cell and is present on its cell surface (second cell surface antigen), and a third antibody that specifically binds to a cell surface antigen that is expressed by a tumor cell and is present on its cell surface and that is different than the second cell surface antigen (third cell surface antigen). Each of the antibodies is labeled (indirectly or directly), for example, with a different fluorophore such that the emission spectra of each fluorophore is distinguishable from the others and from the fluorescent dye (MDR dye).

In one embodiment, the methods described herein include an agent that is known to inhibit MDR activity and that is included in the assay to indicate that efflux of the dye (and thus efflux of a chemotherapeutic agent from a tumor cell) is associated with MDR activity in the cell. Illustrative agents include, but are not limited to verapamil, quinine, progesterone, tamoxifen, atropine, forskolin, and cyclosporin (see, e.g., U.S. Pat. No. 6,660,725 and references therein for a description of compounds that block transporter or flippase activity), and Enniatin B,. Fumitremorgin C, JS-2190, MK-571 sodium salt, PGP-4008, Probenecid, Reversin 121, Reversin 205, WP631 dihydrochloride, WP631 dimethanesulfonate, verapamil (all commercially available; see e.g., Alexis Platform, San Diego, Calif.), XR9576 (QLT, Inc. Vancouver, Calif.). See also M. Zloh and S. Gibbons, 2004, Int. J. Mol. Sci. 5:37-47. As would be recognized by the skilled artisan, any agent that inhibits MDR activity is useful in the context of the present invention and is therefore contemplated herein. In certain other embodiments as described herein, the agent is a candidate agent that is being screened for its capability to block or inhibit efflux of a chemotherapeutic agent from a tumor cell or to inhibit or block MDR activity, such as transporter activity, in a tumor cell. A candidate agent may be any of the agents known in the art that inhibit MDR, such as those agents that block or inhibit p-glycoprotein transport activity as described herein. Alternatively, the candidate agent may be a peptide, polypeptide, nucleic acid, lipid, or small molecule that is provided in a “library” or collection of compounds, compositions, or molecules. Molecules include compounds known in the art as “small molecules” that have molecular weights less than 10⁵ daltons, less than 10⁴ daltons, or less than 10³ daltons. Candidate agents further may be provided as members of a combinatorial library, which includes synthetic agents prepared according to a plurality of predetermined chemical reactions performed in a plurality of reaction vessels. The resulting products comprise a library that can be screened and then followed by iterative selection and synthesis procedures to provide, for example, a synthetic combinatorial library of peptides (see, e.g., PCT/US91/08694, PCT/US91/04666) or other compositions that may include small molecules as provided herein (see, e.g. PCT/US94/08542, U.S. Pat. No. 5,798,035, U.S. Pat. No. 5,789,172, U.S. Pat. No. 5,751,629). Those having ordinary skill in the art will appreciate that a diverse assortment of such libraries may be prepared by a skilled artisan according to established procedures.

Fluorescence Activated Cell Sorting

Approaches for classifying and characterizing hematopoietic cancers include determination of aberrant antigen expression in neoplastic disease, which expression may be categorized into one or more of lineage infidelity, maturational asynchrony, antigenic absence and quantitative abnormalities, as described by Loken et al. (2000). Particularly useful in such approaches has been the use of flow cytometry technology, including multidimensional flow cytometry (see, e.g., Terstappen et al., Anal. Cell. Pathol. 2:229-240 (1990); Terstappen et al., In Progress in Cytometry-Vol. II: Flow and Image, Jansen, A. (Ed.), Becton-Dickinson, Erembodegen-Aalst, Belgium (1989) to determine hematopoietic lineage, maturational stage, and similarity of cells in a patient biopsy sample to well described hematopathologic malignancies (e.g., Jennings et al., Blood 90:2863 (1997)). Such information may be useful, inter alia, for identifying appropriate therapeutic strategies suitable for treating certain malignancies, for distinguishing normal regenerating hematopoietic blast cells from malignant cells (see, e.g., Wells et al., Am. J. Clin. Pathol. 110:84 (1998); Wells et al., Leukemia 12:2015 (1998); Shulman et al., Am. J. Clin. Pathol. 112:513 (1999)), and/or for detecting relapse.

Briefly, such multidimensional flow cytometric methodologies permit characterization of each cell in a single-cell suspension by quantitative determination of multiple molecular markers in combination with forward light scattering (a measurement of cell size) and right-angle light scattering (a measurement of cell granularity). Typically, multiple discrete fluorophores are used to directly or indirectly label monoclonal antibodies specific for various cell surface markers, and cytometers are equipped with light sources (e.g., HeNe or Ar lasers with narrow band-pass filters) and detectors (e.g., sensors with photomultiplier tubes) capable of accurately measuring signal levels generated from defined portions of the fluorescence excitation/emission spectra of the fluorophores. Using a commercially available fluorescence activated cell sorter, such as the FACSCalibur™ or FACSVantage™ (Becton Dickinson, San Jose, Calif.), the EPICS® ALTRA™ (Beckman Coulter, Fullerton, Calif.) or the MoFlo® sorter (DakoCytomation, Inc., Carpinteria, Calif.), cell populations can be sorted into purified fractions. As would be readily recognized by the skilled artisan, a wide variety of cell populations can be differentiated and sorted using immunofluorescence and flow cytometry.

The number of molecular markers that may be simultaneously quantified is limited only by the number of spectrally separable detectable labels that are available and that can be detected by the particular instrumentation employed. The flow cytometer analyzes the heterogeneous cell population one cell at a time and can classify the cells based on the binding of the immunofluorescent monoclonal antibody and the light scattering properties of each cell (see, for example, Immunol. Today 21:383-90 (2000)). By combining the light scatter properties with, for example, two or more colors of immunofluorescent staining, cell populations of interest may be unambiguously identified.

The technique of immunofluorescent staining is well known and can be performed according to any one of a variety of protocols, such as those described in Current Protocols in Cytometry (John Wiley & Sons, NY, NY, Eds. J. Paul Robinson, et al.). Generally, a biological sample, such as peripheral blood, bone marrow, lymph node tissue, cord blood, thymus tissue, tissue from a site of infection, spleen tissue, tumor tissue, and the like, is collected from a subject and cells are isolated therefrom using techniques known in the art. In one embodiment, blood or bone marrow is collected from a subject and any mature erythrocytes are lysed using a buffer, such as buffered NH₄Cl. The remaining leukocytes are washed and then incubated with antibodies (e.g., monoclonal antibodies, or antigen-binding fragments thereof) conjugated to any of a variety of fluorophores known in the art (see for example, http://www.glenspectra.co.uk/glen/filters/fffluorpn.htm or http://cellscience.bio-rad.com/fluorescence/fluorophoradata.htm). Representative fluorophores in this context include, but are not limited to, FITC (fluorescein isothiocyante), R-phycoerythrin (PE), Allophycocyanin (APC), Cy7®, and Texas Red.

By way of background, all hematopoietic cells are believed to derive from common multipotent hematopoietic stem cells that differentiate according to one of the specific hematopoietic lineages (e.g., erythroid, myeloid, or lymphoid) and that subsequently undergo a maturation process, as reviewed by Loken et al. (in Immunophenotyping, pages 133-160, Stewart and Nicholson, (eds.), Wiley-Liss, Inc., New York (2000)). Accordingly, an ever-expanding set of marker molecules has been compiled, and includes cell surface protein and glycoprotein antigens defined using monoclonal antibodies, cytoplasmic or nuclear proteins, and/or carbohydrates identified by chemical, biochemical, or immunochemical reactivities. These markers permit characterization of individual cells according to hematopoietic lineage and maturational state. For example, a person having ordinary skill in the art will be readily familiar with any of the hematopoietic differentiation antigens or other cell surface markers, including those assigned “cluster designation” (CD) numbers, which are systematically numbered hematopoietic cell surface marker antigens identified using monoclonal antibodies according to conventions described in publications of several international workshops (e.g., Leukocyte Typing-VI, K. Kishimoto et al., (eds.), Garland Publishing, NY (1998); Leukocyte Typing-V, Oxford Univ. Press, Oxford, UK (1995); Leukocyte Typing-IV, W. Knapp et al. (eds.), Oxford Univ. Press, Oxford, UK (1989); CD index at http://www.ncbi.nlm.nih.gov/PROW/guide/45277084.html; Current Protocols in Immunology, John Wiley & Sons, NY) have proven to be extremely useful in classification schemes for hematopoietic malignancies. These markers are also useful for distinguishing normal hematopoietic from malignant cells (tumor cells).

A wide variety of antibodies known in the art, and specific antibodies generated using techniques well known in the art, are useful for detecting cell markers. Generally, the antibodies for use in the methods described herein are specific for a cell marker of interest, such as any of the CD cell surface markers (also called CD cell surface antigens), cytokines, adhesion proteins, developmental cell surface markers, tumor antigens, or other proteins expressed by a cell population of interest. An antibody that binds specifically to a protein expressed by a normal cell, a tumor cell, or both a normal cell and tumor cell is useful in the context of this invention.

Methods as described herein for detecting cells that have MDR activity include detecting MDR activity in tumor cells such as leukemia and lymphoma cells that may be present in a biological sample (such as bone marrow or blood). Such tumor cells include leukemic blast cells, acute myelogenous leukemia cells, a chronic myelogenous leukemia cells, an acute lymphocytic leukemia cells, and a chronic lymphocytic leukemia cells, and include lymphoma cells such as Hodgkin's lymphoma cells, a non-Hodgkin's lymphoma cells a T lymphoblastoid lymphoma cells, and a B lymphoblastoid lymphoma cells.

Any normal cell that is present in the biological sample to be analyzed, that expresses MDR proteins, and that can be distinguished from the malignant cells by at least one cell surface marker, is useful to determine MDR activity in normal cells (e.g., as an internal control) in the present invention. In certain embodiments, the normal cell detected in a biological sample (e.g., bone marrow or blood) is a natural killer (NK) cell. NK cells may be detected, for example, with with an antibody that binds specifically to CD11b, an antibody that binds specifically to CD56, or with an antibody that binds specifically to CD16. A tumor cell (malignant cell) that may be detected in the biological sample may be, for example, a leukemic blast cell. Such a leukemic blast cell may be detected using at least one antibody that specifically binds to a cell surface antigen or may be detected using two or more antibodies that each specifically bind to different cell surface antigens. Cell surface antigens that are expressed on a tumor cell, such as a leukemic blast cell, include for example, CD34, CD45, CD33, CD13, and CD38. Note that antibodies useful in the present invention should survive incubation at 37 degrees C. Thus, in certain embodiments, antibodies to markers that do not work well at 37 degrees C. alone may be useful at 37 degrees C. with stabilization.

A detectable signal is generated by contacting cells with a detectably labeled antibody (directly labeled or indirectly labeled) capable of specifically binding to a cell surface antigen under conditions and for a time sufficient to detect such binding. The level of a signal so generated may be compared to a control level of the detectable signal generated by contacting the cells with a detectably labeled ligand that is not capable of specifically binding to the cells, under otherwise similar conditions. Such determination of the amount of cell surface antigen may be accomplished by any of a number of procedures with which those having ordinary skill in the art will be readily familiar, including but not limited to flow cytometry (including flow immunocytometry and flow immunocytofluorimetry), immunofluorescence microscopy (including laser-scanning confocal microscopy), immunoelectron microscopy and other methodologies known to the art for quantifying specific receptors in individual cells.

As described herein, a cell surface marker antigen, may include any molecule or structure that naturally or as the result of genetic engineering, spontaneous or artificially induced mutation, pathogenetic mechanism, pharmacological intervention, chemical or physical intervention or insult, or any other biological process, may be detectably expressed on a cell surface. Such cell surface marker antigens typically are proteins, glycoproteins, proteoglycans, proteolipids, or glycolipids, but may also exist as carbohydrates, lipids, nucleic acids, or other classes of molecules. Particularly useful for the methods, and as described herein, are cell surface marker antigens of hematopoietic cells and tissues, such as those marker antigens that have been characterized through the use of monoclonal antibodies as described herein and known in the art.

In flow cytometric embodiments, cell types, including different types of normal hematopoietic cells, within a plurality of cells may be characterized by a set of forward light scattering criteria (e.g., a range of detected signal values defining cells within a particular size range) and/or right-angle light scattering criteria (e.g., a range of detected signal values defining cells having a particular degree of granularity) and/or a set of fluorescence intensity criteria for one or more particular cell surface antigen markers as provided herein (e.g., a range of detected signal values defining cells having a particular amount of the marker expressed), to derive a subset or subpopulation of cells in a sample comprising a plurality of cells.

Examples of cell selection criteria for use in cell selection profiles according to non-flow cytometric embodiments include determination of cellular morphology according to any of a wide variety of known anatomical, ultrastructural, histochemical, immunochemical, and/or biochemical parameters or the like; such criteria may in certain embodiments also include functional or selectable properties of normal cells or tumor cells that permit identification of such a selected population. For instance, such properties may include cell viability under defined conditions, cell adhesion to a particular substrate under defined conditions, cell motility, or cellular expression of one or more selection markers or reporter genes, or other detectable traits that will vary as a function of the particular cell type and biological system under consideration, are which are known to those familiar with the art.

Biological Sample

According to the present invention, a cell surface marker may be detected in a biological sample from a subject or biological source. Biological samples may be provided by obtaining a blood sample, biopsy specimen, tissue explant, organ culture, biological fluid or any other tissue or cell preparation from a subject or a biological source. The subject or biological source may be a human or non-human animal, a primary cell culture or culture-adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines and the like. In certain embodiments, the subject or biological source may have a malignant condition or be suspected of having or being at risk for having a malignant condition.

In certain embodiments the biological sample is a biological fluid or a liquid containing a plurality of cells, in particular embodiments the plurality of cells are intact hematopoietic cells in suspension, including but not limited to bone marrow cells, blood cells, lymph node cells, thymus-derived cells, or other cells of the several hematopoietic lineages. Biological fluids are typically liquids at physiological temperatures and may include naturally occurring fluids present in, withdrawn from, expressed or otherwise extracted from a subject or biological source. Certain biological fluids derive from particular tissues, organs, or localized regions, and certain other biological fluids may be more globally or systemically situated in a subject or biological source. Examples of biological fluids include blood, bone marrow, serum and serosal fluids, plasma, lymph, urine, cerebrospinal fluid, saliva, mucosal secretions of the secretory tissues and organs, semen, vaginal secretions, ascites fluids such as those associated with non-solid tumors, fluids of the pleural, pericardial, peritoneal, abdominal and other body cavities, and the like.

Biological fluids may also include liquid solutions contacted with a subject or biological source, for example, cell and organ culture medium including cell or organ conditioned medium, lavage fluids and the like. In a particular embodiment the biological sample is blood or a blood-derived fraction, and in certain other particular embodiments the biological sample is bone marrow or a marrow-derived fraction. In certain other embodiments determining a level of a detectable signal derived from an intracellular marker may be desirable, wherein the intracellular marker includes but is not limited to a cytosolic marker, a cytoplasmic marker, or an organellar marker, for example, an intracellular molecule associated with an organellar membrane, or an organelle, or an organellar substance, such as may be found associated with cytoplasmic granules. Techniques for detecting such intracellular markers are known in the art (e.g., intracellular cytokine assays and the like).

Malignant Condition

The presence of a malignant condition in a subject refers to the presence of dysplastic, cancerous, and/or transformed cells in the subject, including, for example neoplastic, tumor, non-contact inhibited, or oncogenically transformed cells, or the like. By way of illustration and not limitation, in the context of the present invention a malignant condition may refer further to the presence in a subject of cancer cells (tumor cells or malignant cells) of hematopoietic origin, including malignancies of multipotent hematopoietic stem cells or of cells of any differentiative or maturational stage derived therefrom. According to related embodiments, malignant cells of hematopoietic origin need not necessarily correspond exactly to any particular differentiative or maturational stage of a non-malignant hematopoietic cell. Without wishing to be bound by theory, the majority of malignant cells present in many hematopoietic malignant conditions do not correspond strictly to normal (i.e., non-transformed) counterparts when comparisons are made using a sufficient number of phenotypic markers (e.g., cell surface marker antigens, intracellular markers, etc.) (see, e.g., Loken et al. In Immunophenotyping, C. Stewart and J. K. A. Nicholson, (eds.), Wiley-Liss, Inc., New York, pages 133-160 (2000) and references cited therein).

In the certain embodiments of the invention, malignant cells (tumor cells), the presence of which signifies the presence of a malignant condition, are leukemic, including transformed cells of myeloid lineage. Criteria for classifying a malignancy as leukemia are described herein and are well known in the art (see, e.g., Concensus Conference in Hematologic Oncology (1997); World Health Organization Hematology/Oncology Classification (2000); and references cited therein) as are the establishment and characterization of leukemic cell lines from primary and metastatic tumors. In other embodiments, the malignant condition may be any of a wide variety of other hematologic malignancies (e.g., leukemias, lymphomas, myelomas, etc.).

Detectable Signal

As provided herein and as known in the art, a variety of physicochemical and/or biochemical properties may be usefully exploited to detect and quantify a signal from a biological sample as provided herein, or a portion or component thereof such as a cell, cell surface marker antigen, or a detectably labeled ligand such as an antibody. These properties are selected according to the type of sample, and are also based on the nature of the parameter to be determined through signal detection, so long as compositions and methods, including detection instruments, are available for detecting, quantifying or otherwise monitoring a desired signal. For example, a signal that may be detected according to the methods of the present invention may be one or more of (or a combination of) an electrical signal (e.g., an amperometric or voltometric signal, a radio frequency or magnetic signal, a digital signal, or the like); an optical or photometric signal, such as a spectrophotometric, calorimetric, fluorimetric, densitometric, polarimetric or refractometric signal or the like; and/or a chemical, immunochemical or other biochemical signal, such as a radiometric, phosphorescent, luminescent, spectroscopic, surface plasmon resonance, enzymatic, or immunometric signal. The person having ordinary skill in the art will be familiar with these and other detectable signals and can further readily and without undue experimentation select an appropriate detectable label capable of generating a detectable signal as provided herein, for use according to the methods of the claimed invention.

Accordingly, and in certain embodiments of the present invention, a detectable signal comprises a fluorescent signal detected in a biological sample, which includes any fluorescent properties naturally present in the sample and which also may include any fluorescent properties introduced into the sample as a result of artificial manipulations thereof. For example, a fluorescent signal may include inherent fluorescent properties of a biological sample that is examined under appropriate conditions and with which those skilled in the art will be familiar, and which may be referred to as “autofluorescence.” A fluorescent signal may also, for example, include fluorescent properties that are artificially introduced into the sample, such as those provided by a detectably labeled molecule comprising a directly (e.g., covalently) or indirectly (e.g., noncovalently) attached fluorophore that is combined with the sample. Accordingly, a detectable signal as provided herein may be referred to as a fluorescent signal and may in certain embodiments comprise either or both of an autofluorescent signal and a fluorescent signal generated, for instance, by a detectably labeled antibody.

Those familiar with the art will appreciate that in certain preferred embodiments of the present invention, determination of a detectable signal of interest is usefully accompanied by comparison of such level to an appropriate “control” level of the detectable signal, in order to more accurately determine the level of the signal of interest. Thus, for example, when the detectable signal is a fluorescent signal, and when the level of the fluorescent signal generated by at least one detectably labeled antibody specific for a cell surface marker antigen is to be determined, the invention contemplates comparing a control level of a detectable fluorescence signal to the level generated by the detectably labeled antibody. In certain embodiments the control level of fluorescence may be derived from autofluorescence in the sample, and in certain embodiments the control level of fluorescence may be non-specific background fluorescence (if any) generated by a negative control antibody having an irrelevant specificity, such that the antibody is detectably labeled but does not specifically bind to the sample. In certain embodiments the control level of fluorescence may be the sum of such detectable autofluorescence and non-specific background fluorescence signals. While exemplified herein in the context of a control level that is a level of a detectable fluorescent signal, the invention is not intended to be so limited, such that variations on what may be an appropriate control level of a fluorescent signal or of any other detectable signals are within the scope and spirit of the invention.

The methodology employed for detection of the signal may also vary according to the particular sample that is investigated, the nature of the signal being detected and the instrumentation available. In one embodiment, and as described herein, detectable signals are determined through the use of flow cytometry, which may include any of a number of techniques for monitoring one or a plurality of cellular parameters at the level of data collected from a plurality of individual cells in a single cell suspension that is passed through a detector capable of discerning detectable signal(s) from each cell (see, e.g., Loken et al. In Immunophenotyping, C. Stewart and J. K. A. Nicholson, (eds.), 2000 Wiley-Liss, Inc., New York, pages 133-160, and references cited therein; see also Current Protocols in Cytometry, 1997 John Wiley & Sons, New York). Particularly preferred is flow immunocytofluorimetry, in which data from detectable signals that relate to physical properties that permit differential quantitative characterization of cells according to size (e.g., forward light scatter) and granularity (e.g., right-angle or “side” light scatter) are combined with data from one or more discrete fluorescence intensity signals generated by one or more detectably labeled antibodies that are directly or indirectly labeled with one or more spectrally distinguishable fluorophores, to generate multiparameter profiles for each cell analyzed. Thus, in such an embodiment, detectable signals are determined using flow immunocytofluorimetric analysis of various hematopoietic cell preparations or portions thereof (e.g., defined subpopulations) including the use of well-established cell marker antigen systems. These and other useful methodologies may be found, for example, in Rose et al. (eds.), Manual of Clinical Laboratory Immunology, 5^{th} Ed., American Society of Microbiology, Washington, D.C. (1997).

Other methodologies and instrumentation may be additionally or alternatively employed according to the subject invention methods, including but not limited to laser scanning confocal microscopy, immunofluorescence microscopy, immunocytochemistry, immunoelectron microscopy or morphometric analysis, which may be preferably combined with any of various known image analysis processing techniques, and which are desirably performed in an automated fashion. These and other cytometric techniques may be within the scope of the methods provided herein, so long as determination of a detectable signal from individual cells can be achieved. As noted above, in most preferred embodiments of the present invention a detectable signal is determined using flow cytometry.

Antibodies

Ligands that are capable of specifically binding to a cell surface marker or antigen, and that can be detectably labeled, include antibodies as well as other ligands such as a counterreceptor, a hormone, a protein, a lectin, a glycoprotein, a polypeptide or peptide or the like, or any other binding molecule that participates in a specific binding interaction with a cognate binding partner such as a cell surface antigen, a counterligand or the like. In particular embodiments, the ligand is an antibody that is specific for a desired antigen, such as a cell surface marker antigen as provided herein (or for an intracellular marker) and is readily generated as a monoclonal antibody or as polyclonal antisera, or may be produced as a genetically engineered immunoglobulin (Ig) that is designed to have desirable properties using methods well known in the art. For example, by way of illustration and not limitation, antibodies may include recombinant Igs, chimeric fusion proteins having immunoglobulin derived sequences, or “humanized” antibodies (see, e.g., U.S. Pat. Nos. 5,693,762; 5,585,089; 4,816,567; 5,225,539; 5,530,101; and references cited therein) that may all be used for detection of a cell surface marker antigen according to the invention. Many such antibodies have been disclosed and are available from specific sources or may be prepared according to well established procedures, including by immunization with particular cell lines or tissue sources, as described, for example, in publications from several international workshops (e.g., Leukocyte Typing-VI, 1998 K. Kishimoto et al., (eds.), Garland Publishing, NY; Leukocyte Typing-V, 1995 Oxford Univ. Press, Oxford, UK; Leukocyte Typing-IV, 1989 W. Knapp et al. (eds.), Oxford Univ. Press, Oxford, UK; etc.) and references cited therein. Various other sources of antibodies specific for marker antigens that may be usefully determined according to the methods provided by the present invention are known to those having ordinary skill in the art, as provided, for example, in Linscott's directory of antibodies (www.linscottsdirectory.com) or as may be found at any number of other sources.

The term antibody or antibodies includes polyclonal antibodies, monoclonal antibodies, and fragments thereof such as F(ab′)₂, and Fab and Fab′ fragments, as well as any naturally occurring or recombinantly produced binding partners, which are molecules that specifically bind a cell surface marker antigen or an intracellular marker, for example, CD5, CD7, CD11b, CD13, CD14, CD15, CD16, CD19, CD20, CD33, CD34, CD36, CD38, CD45 and CD56, and in certain particular embodiments, CD56 and CD34, CD56 and CD45, or CD56 and CD34 and CD45.

As used herein, an antibody that specifically binds to a cell surface antigen reacts at a detectable level with the antigen, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and an antibody specifically binds to a cell surface antigen if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of binding partners or antibodies can be readily determined using conventional techniques, for example, those described by Scatchard et al. (Ann. N.Y. Acad Sci. USA 51:660 (1949)) and by surface plasmon resonance (SPR; BIAcore™, Biosensor, Piscataway, N.J.) (see, e.g., Wolff et al., Cancer Res. 53:2560-2565 (1993)).

Antibodies useful in the present invention are generally commercially available through a number of sources familiar to the skilled artisan. Antibodies may also generally be prepared by any of a variety of techniques known to those having ordinary skill in the art (see, e.g., Harlow and Lane, Antibodies: A Laboratory Manual, Cold Spring Harbor Laboratory, 1988), and monoclonal antibodies may be particularly preferred in certain embodiments of the present invention. Monoclonal antibodies (e.g., a mouse, human, rat, hamster, camel, chicken, goat monoclonal antibody) specific for CD45 or CD34 or other cell surface antigens, or variants thereof may be prepared, for example, using the technique of Kohler and Milstein (Nature, 256:495-497 (1976); Eur. J. Immunol. 6:511-519 (1975)) and improvements thereto (see also, e.g, Coligan et al. (eds.), Current Protocols in Immunology, 1:2.5.1-2.6.7 (John Wiley & Sons 1991); U.S. Pat. Nos. 4,902,614, 4,543,439, and 4,411,993; Monoclonal Antibodies, Hybridomas: A New Dimension in Biological Analyses, Plenum Press, Kennett et al. (eds.) (1980); and Antibodies: A Laboratory Manual, Harlow and Lane (eds.), Cold Spring Harbor Laboratory Press (1988); WO 92/02551; U.S. Pat. No. 5,627,052; Babcook et al., Proc. Natl. Acad. Sci. USA 93:7843-48 (1996); Green et al., Nature Genet. 7:13, 1994; Lonberg et al., Nature 368:856, 1994; Taylor et al., Int. Immun. 6:579 (1994); U.S. Pat. No. 5,877,397; Bruggemann et al., Curr. Opin. Biotechnol. 8:455-58 (1997); Jakobovits et al., Ann. N.Y. Acad. Sci. 764:525-35 (1995)).

An antibody that specifically binds to a cell surface antigen that may be used in the methods described herein includes a chimeric antibody. A chimeric antibody has at least one constant region domain derived from a first mammalian species and at least one variable region domain derived from a second, distinct mammalian species. Chimeric and humanized antibodies may be prepared by a variety of methods practiced by persons skilled in the art (see, e.g., Morrison et al., Proc. Natl. Acad. Sci. USA, 81:6851-55 (1984); Shin et al., Methods Enzymol. 178:459-76 (1989); Walls et al., Nucleic Acids Res. 21:2921-29 (1993)). An antibody as used herein also includes a humanized antibody, which may include a plurality of CDRs (complementarity determining regions) derived from an immunoglobulin of a non-human mammalian species, at least one human variable framework region, and at least one human immunoglobulin constant region (see, e.g., Padlan et al., FASEB 9:133-39 (1995); Chothia et al., Nature, 342:377-383 (1989); Bajorath et al., Ther. Immunol. 2:95-103 (1995); EP-0578515-A3); Davies et al., Ann. Rev. Biochem. 59:439-73 (1990)).

Within certain embodiments, the use of antigen-binding fragments such as F(ab′)₂ Fab, Fab′, Fv, and Fd, of antibodies may be desired. Antibody fragments can be obtained, for example, by proteolytic hydrolysis of the antibody or can be prepared by recombinant methods practiced in the art. See, e.g., Weir, D. M., Handbook of Experimental Immunology, 1986, Blackwell Scientific, Boston. As an illustration, antibody fragments can be produced by enzymatic cleavage of antibodies with pepsin to provide a fragment denoted F(ab′)₂. This fragment can be further cleaved using a thiol reducing agent to produce an Fab′ monovalent fragment. Optionally, the cleavage reaction can be performed using a blocking group for the sulfhydryl groups that result from cleavage of disulfide linkages. As an alternative, an enzymatic cleavage of an antibody using papain produces two monovalent Fab fragments and an Fc fragment. The cleaved Fc fragments may be separated from the antigen-binding fragment by affinity chromatography (e.g., on immobilized protein A columns or immobilized Fc specific reagent columns) using standard techniques. (See, e.g., U.S. Pat. No. 4,331,647; Nisonoff et al., Arch. Biochem. Biophys. 89:230, 1960; Porter,. Biochem. J. 73:119, 1959; Edelman et al., in Methods in Enzymology 1:422 (Academic Press 1967); Weir, Handbook of Experimental Immunology, Blackwell Scientific, Boston (1986)).

An antibody fragment may also be any synthetic or genetically engineered protein that acts like an antibody in that it binds to a specific antigen to form a complex. For example, antibody fragments include isolated fragments consisting of the light chain variable region, Fv fragments consisting of the variable regions of the heavy and light chains, recombinant single chain polypeptide molecules in which light and heavy variable regions are connected by a peptide linker (scFv proteins), and minimal recognition units consisting of the amino acid residues that mimic the hypervariable region. The antibody of the present invention preferably comprises at least one variable region domain.

Multifunctional fusion proteins having specific binding affinities for pre-selected antigens by virtue of immunoglobulin V-region domains encoded by DNA sequences linked in-frame to sequences encoding various effector proteins are known in the art, for example, as disclosed in EP-B1-0318554, U.S. Pat. No. 5,132,405, U.S. Pat. No. 5,091,513 and U.S. Pat. No. 5,476,786. Such effector proteins include polypeptide domains that may be used to detect binding of the fusion protein by any of a variety of techniques with which those skilled in the art will be familiar, including but not limited to a biotin mimetic sequence (see, e.g., Luo et al., 1998 J. Biotechnol. 65:225 and references cited therein), direct covalent modification with a detectable labeling moiety, non-covalent binding to a specific labeled reporter molecule, enzymatic modification of a detectable substrate or immobilization (covalent or non-covalent) on a solid-phase support.

Single chain antibodies (scFv) for use in the methods described herein may also be generated and selected by a method such as phage display (see, e.g., U.S. Pat. No. 5,223,409; Schlebusch et al., 1997 Hybridoma 16:47; and references cited therein). Briefly, in this method, DNA sequences are inserted into the gene III or gene VIII gene of a filamentous phage, such as M13. Several vectors with multicloning sites have been developed for insertion (McLafferty et al., Gene 128:29-36, 1993; Scott and Smith, Science 249:386-390, 1990; Smith and Scott. Methods Enzymol. 217:228-257, 1993). The inserted DNA sequences may be randomly generated or may be variants of a known binding domain for binding to an antigen of interest for use according to the present invention, for example, a cell surface marker antigen. The DNA sequence of the insert in the binding phage that encodes a binding domain of interest is then determined. Once the predicted amino acid sequence of the binding peptide is known, sufficient peptide for use herein as an antibody specific for an antigen of interest, such as a cell surface marker antigen, may be made either by recombinant procedures or synthetically.

In certain embodiments, the antibody that is specific for a cell surface marker antigen may contain or be directly detectably labeled with a detectable reporter moiety or label such as a dye, luminescent group, fluorescent group, or biotin, or the like. In particularly preferred embodiments the detectable reporter moiety is a fluorescent reporter moiety (fluorophore) such as fluorescein (such as fluorescein isothiocyanate), phycoerythrin, rhodamine, Texas Red, BODIPY, Allophycocyanin (APC), Cy7®, or any of a wide variety of fluorescent reporter molecules known to the art for such purposes, for example, those disclosed in Haugland (Handbook of Fluorescent Probes and Research Chemicals-Sixth Ed., Molecular Probes, Eugene, Oreg. (1996)) or available from Becton Dickinson, Inc. (San Jose, Calif.), Coulter Instruments (Hialeah, Fla.) or a variety of other manufacturers and suppliers with which those having ordinary skill in the art will be readily familiar. The detectably labeled antibody is prepared in a manner that provides predictable, reproducible, and quantifiable stoichiometry with respect to the ligand molecule and the detectable labeling moiety. A variety of conjugation methods and procedures for determining the stoichiometry of a detectable labeling moiety on a ligand are known to those having familiarity with the art (e.g., Weir, Handbook of Experimental Immunology, 1986, Blackwell Scientific, Boston; see also Current Protocols in Cytometry, 1997 John Wiley & Sons, New York).

In certain other embodiments, the antibody that is capable of specifically binding to a cell surface marker antigen does not itself contain (or is not directly labeled with) such a detectable reporter moiety, but is instead detectably labeled by indirect methods well known to those having ordinary skill in the art, for example, a labeled secondary antibody or other suitable indirect labeling reagent (e.g., labeled avidin or streptavidin for use with a primary antibody that is biotinylated, or labeled protein A, etc.). Those familiar with the art can readily and without undue experimentation devise appropriate configurations of unlabeled or labeled specific antibodies for indirect or direct immunofluorescence, respectively, and, optionally, labeled secondary reagents for indirect immunofluorescence, any combination of which is encompassed within the meaning of an antibody that is “detectably labeled” as provided herein.

Although specific embodiments are described herein, certain variations on the present disclosure are within the scope and spirit of the invention. The following Examples are offered by way of illustration and not by way of limitation.

### EXAMPLES

### Example 1

### Detection of Multi-Drug Resistance in Leukemic Cells and NK Cells and Determination of the Effect of an Agent that Inhibits Multi-Drug Resistance Activity

Flow cytometric analysis is performed on a bone marrow aspirate using four-color combinations of reagents.

An antibody that specifically binds to CD11b, an antibody that specifically binds to CD45, and an antibody that specifically binds to CD34 are combined with the MDR dye (e.g., DiOC) and 100 μl of whole bone marrow (or blood) in a plastic test tube and incubated in the dark at 37° C. for 20 minutes. Each antibody is labeled with a different flurophore that will not interfere with the emission spectra of the MDR dye. NK cells (natural killer cells) express CD11b and leukemic blast cells express CD45 and CD34.

The mature RBC are then lysed and the MDR dye that is not taken up by any cell is removed in a single step by adding 3.5 ml warm (37° C.)-buffered NH₄Cl for 5 minutes. 100 ul of fetal calf serum (FCS) is added to the bottom of the tube to form a discontinuous gradient. The cells are pelleted by centrifugation at 350×g for 5 minutes. The supernatant is removed and the cells are resuspended in 1.5 ml tissue culture media (RPMI1640 or DMEM) containing 20% FCS.

The cell suspension is divided into 3 aliquots. The first aliquot (#1) is incubated on ice (0° C.). A second aliquot (#2) is incubated at 37° C. for 90 minutes. To a third aliquot (#3), an MDR inhibitor drug is added and then incubated at 37° C. for 90 minutes.

After the 90 minute incubation, the fluorescence intensities of cells in the 3 tubes are compared by analyzing the specimens on a flow cytometer (4 color). Thus, the results can be conveniently broken down into the following groups based on cell populations and test conditions:

1N (normal cells, e.g., NK cells bound by the anti-CD11b antibody, first aliquot)

2N (normal cells, e.g., NK cells bound by the anti-CD11b antibody, second aliquot)

3N (normal cells e.g., NK cells bound by the anti-CD11b antibody, third aliquot).

1C (leukemic blast cells bound by anti CD45 and anti-CD34 antibodies, first aliquot)

2C (leukemic blast cells bound by anti CD45 and anti-CD34 antibodies, second aliquot)

3C (leukemic blast cells bound by anti CD45 and anti-CD34 antibodies, third aliquot)

A decrease in the MDR dye detected in CD11b+ NK cells (2N vs 1N) serves as an internal control to show that the method detects MDR activity. A decrease in MDR dye in the leukemic blast cells (2C vs 1C) identified by CD45 and CD34 shows that the leukemic cells have MDR activity. A relative proportion between MDR activity detected in NK cells and MDR activity detected in the leukemic cells is used for standardizing the assay (i.e., 2N vs 2C, 2N/2C).

In a similar manner, inhibition of the MDR pump by the MDR inhibitor drug is determined by comparing the MDR dye in NK cells in group 3N vs group 1N and group 3N vs 2N. Similarly, inhibition of MDR activity in the leukemic cells is quantified by comparing the amounts of MDR between group 3C vs group 1C and group 3C vs group 2C.

### Example 2

### Detection of Multi-Drug Resistance in Leukemic Cells and NK Cells

This example describes the detection of MDR in leukemic cells from a patient with acute myeloid leukemia (AML) using the assay described in Example 1.

Bone marrow cells from a patient with AML were incubated at 37 degrees for 30 minutes with CD11b phycoerythrin, CD45 peridinin chlorophyll protein, CD34 allophycocyanin and DiOC2 (an MDR sensitive dye). Analysis by the flow cytometer using standard techniques (see e.g., Loken M R and Wells D A, Normal Antigen Expression in Hematopoiesis: Basis for Interpreting Leukemia Phenotypes, in Immunophenotyping, Eds Carleton Stewart and Janel K. A. Nicholson, 2000, Wiley-Liss, Inc.) identified 28% abnormal myeloblasts (Blast) based on CD45 and SSC (right angle light scatter).

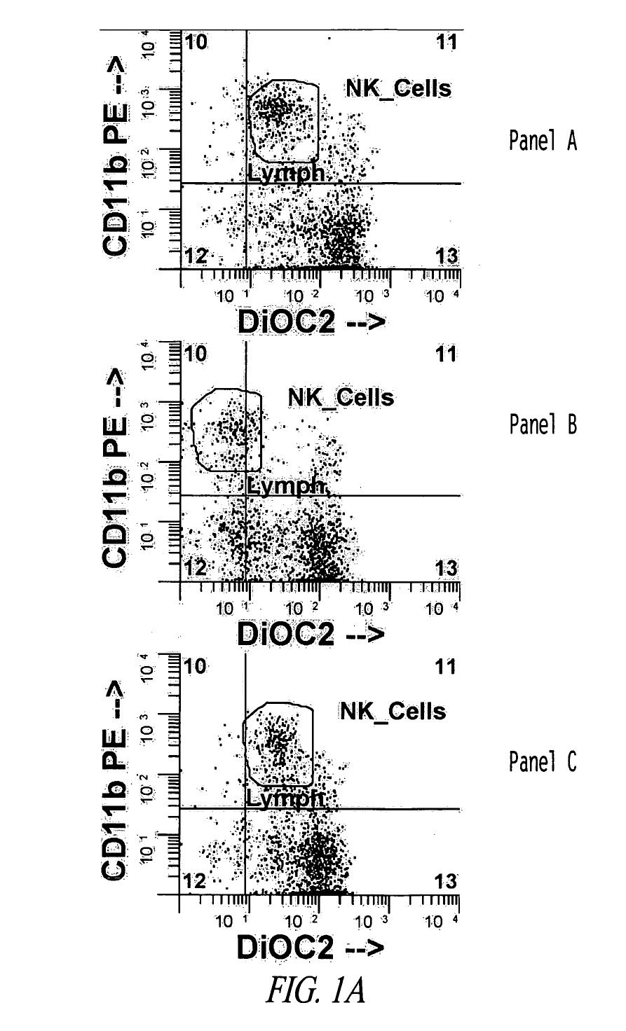

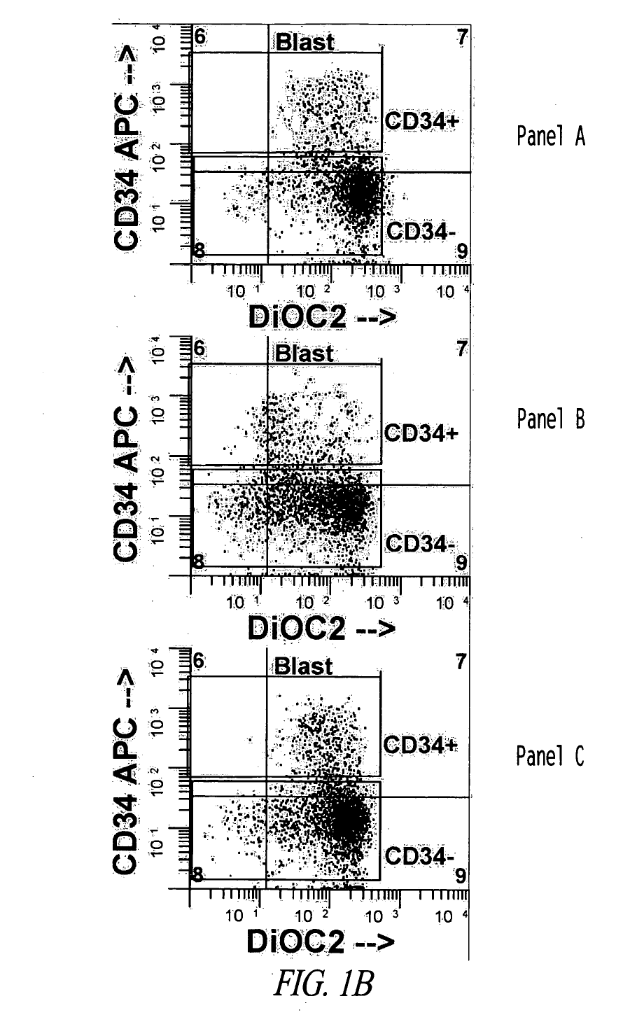

The NK (Natural Killer) cells were identified within the lymphocyte gate based on their expression of CD11b. The abnormal leukemia blasts were identified by CD45 and SSC. Approximately 13% of the leukemia blasts expressed the progenitor cell antigen CD34. As shown in FIGS. 1A and 1B, the bone marrow cells were incubated at 4 degrees C. (Panels A), 37 degrees C. (Panels B), and at 37 degrees C. in the presence of an MDR-1 specific inhibitor (Panels C). The fluorescence intensity of the NK cells depended upon the conditions of incubation (FIG. 1A). After incubation in the cold the mean fluorescence intensity of DiOC2 was 29 (FIG. 1A, panel A). After incubation at 37 degrees the mean fluorescence intensity was 6.4 (FIG. 1A, panel B). With incubation at 37 degrees in the presence of an MDR-1 inhibitor the intensity was 28 (FIG. 1A, panel C). For the leukemic blasts (FIG. 1B), the fluorescence of the DiOC2 was also dependent on the incubation conditions. The CD34 positive leukemic blasts incubated at 4 degrees C. exhibited a DiOC2 fluorescence of 131 (FIG. 1B. panel A), which decreased to 57 after incubation at 37 degrees (FIG. 1B, panel B), and remained high at 101 after incubation at 37 degrees in the presence of the MDR-1 inhibitor (FIG. 1B, panel C). The majority of the CD34 negative cells (e.g., the more mature blasts) did not change in fluorescence as dramatically with the different incubations. However a subset of CD34 negative blast cells did show reduced DiOC2 fluorescence when the cells were incubated at 37 degrees as compared to incubation at 4 degrees or incubation at 37 degrees in the presence of an MDR-1 inhibitor.

In summary, this examples shows that the functional assay described herein can be used to detect multi-drug resistance in leukemic cells using NK cells as an internal control. Further, this demonstrates that this assay can be used for determining the effect of an agent that inhibits multi-drug resistance activity and is useful for determining appropriate therapeutic courses for the treatment of leukemia.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for identifying an agent that inhibits multi-drug resistance activity in a tumor cell comprising:
(a) contacting (i) a biological sample comprising a plurality of cells, (ii) a fluorescent dye that is capable of being transported across a cell membrane, and (iii) at least a first antibody and a second antibody, wherein the first antibody specifically binds to a first cell surface antigen that is present on a normal cell and the second antibody specifically binds to a second cell surface antigen that is present on a tumor cell, and wherein the first antibody is detectably labeled with a first fluorophore and the second antibody is detectably labeled with a second fluorophore, wherein each fluorophore has a distinguishable emission spectra from each other and the dye, under conditions and for a time sufficient to permit interaction among the plurality of cells, the dye, and the antibodies;
(b) isolating the plurality of cells from the biological sample;
(c) retaining a first aliquot of the plurality of cells under conditions that inhibit efflux of the dye from a cell;
(d) retaining a second aliquot of the plurality of cells under conditions and for a time sufficient to permit efflux of the dye from a cell;
(e) retaining a third aliquot of the plurality of cells and contacting the plurality of cells with a candidate agent under conditions and for a time that permit interaction between the cells and the agent;
(f) detecting the level of fluorescence of the dye in cells to which the first antibody binds and in cells to which the second antibody, in each of the three aliquots of cells;
(g) comparing in the first, second, and third aliquots the level of fluorescence of the dye in the plurality of cells to which the first antibody binds, wherein a decreased level of fluorescence of the dye in the second aliquot compared with the level of fluorescence of the dye in the first aliquot indicates efflux of the dye from the normal cell, and wherein an increase in the level of fluorescence of the dye in the third aliquot compared with the level of fluorescence of the dye in the second aliquot indicates that the agent inhibits efflux of the dye from the cell, thereby indicating that the normal cell has multi-drug resistance activity and thereby providing an internal control for determining multi-drug resistance activity; and
(h) comparing in the first, second, and third aliquots the level of fluorescence of the dye in the plurality of cells to which the second antibody binds, wherein a decreased level of fluorescence of the dye in the second aliquot compared with the level of fluorescence of the dye in the first aliquot indicates efflux of the dye from the tumor cell, and wherein an increase in the level of fluorescence of the dye in the third aliquot compared with the level of fluorescence of the dye in the second aliquot indicates that the agent inhibits efflux of the dye from the tumor cell, thereby identifying an agent that inhibits multi-drug resistance activity in a tumor cell.

2. A method for detecting multi-drug resistance activity of a tumor cell comprising:
(a) contacting (i) a biological sample comprising a plurality of cells, (ii) a fluorescent dye that is capable of being transported across a cell membrane, and (iii) at least a first antibody and a second antibody, wherein the first antibody specifically binds to a first cell surface antigen that is present on a normal cell and the second antibody specifically binds to a second cell surface antigen that is present on a tumor cell, and wherein the first antibody is detectably labeled with a first fluorophore and the second antibody is detectably labeled with a second fluorophore, wherein each fluorophore has a distinguishable emission spectra from each other and the dye, under conditions and for a time sufficient to permit interaction among the plurality of cells, the dye, and the antibodies;
(b) isolating the plurality of cells from the biological sample;
(c) retaining a first aliquot of the plurality of cells under conditions that inhibit efflux of the dye from a cell;
(d) retaining a second aliquot of the plurality of cells under conditions and for a time sufficient that permit efflux of the dye from a cell;
(e) contacting a third aliquot of the plurality of cells with an agent that inhibits multi-drug resistance activity, under conditions and for a time that permit interaction between the cells and the agent;
(f) detecting the level of fluorescence of the dye in cells to which the first antibody binds and in cells to which the second antibody, in each of the three aliquots of cells;
(g) comparing in the first, second, and third aliquots the level of fluorescence of the dye in the plurality of cells to which the first antibody binds, wherein a decreased level of dye in the second aliquot compared with the level of fluorescence of the dye in the first aliquot indicates efflux of the dye from the normal cell, and wherein an increase in the level of fluorescence of the dye in the third aliquot compared with the level of fluorescence of the dye in the second aliquot indicates that the agent inhibits efflux of the dye from the normal cell, thereby indicating that the normal cell has multi-drug resistance activity and thereby providing an internal control for determining multi-drug resistance activity; and
(h) comparing in the first, second, and third aliquots the level of fluorescence of the dye in the plurality of cells to which the second antibody binds, wherein a decreased level of fluorescence of the dye in the second aliquot compared with the level of fluorescence of the dye in the first aliquot indicates efflux of the dye from the tumor cell, and wherein an increase in the level of fluorescence of the dye in the third aliquot compared with the level of fluorescence of the dye in the second aliquot indicates that the agent inhibits efflux of the dye from the tumor cell, thereby indicating that the tumor cell has multi-drug resistance activity.

3. A method for monitoring drug resistance activity of a tumor cell from a subject who has a malignant condition, said method comprising:
(a) obtaining a biological sample from a subject who has a malignant condition;
(b) contacting (i) a biological sample comprising a plurality of cells, (ii) a fluorescent dye that is capable of being transported across a cell membrane, and (iii) at least a first antibody and a second antibody, wherein the first antibody specifically binds to a first cell surface antigen that is present on a normal cell and the second antibody specifically binds to a second cell surface antigen that is present on a tumor cell, and wherein the first antibody is detectably labeled with a first fluorophore and the second antibody is detectably labeled with a second fluorophore, wherein each fluorophore has a distinguishable emission spectra from each other and the dye, under conditions and for a time sufficient to permit interaction among the plurality of cells, the dye, and the antibodies;
(c) isolating the plurality of cells from the biological sample;
(d) retaining a first aliquot of the plurality of cells under conditions that inhibit efflux of the dye from a cell;
(e) retaining a second aliquot of the plurality of cells under conditions and for a time sufficient to permit efflux of the dye from a cell;
(f) contacting a third aliquot of the plurality of cells with an agent that inhibits multi-drug resistance activity under conditions and for a time that permit interaction between the cells and the agent;
(g) detecting the level of fluorescence of the dye in cells to which the first antibody binds and in cells to which the second antibody, in each of the three aliquots of cells;
(h) comparing in the first, second, and third aliquots the level of fluorescence of the dye in the plurality of cells to which the first antibody binds, wherein a decreased level of fluorescence of the dye in the second aliquot compared with the level of fluorescence of the dye in the first aliquot indicates efflux of the dye from the normal cell, and wherein an increase in the level of fluorescence of the dye in the third aliquot compared with the level of fluorescence of the dye in the second aliquot indicates that the agent inhibits efflux of the dye from the cell, thereby indicating that the normal cell has multi-drug resistance activity and thereby providing an internal control for determining multi-drug resistance activity; and
(i) comparing in the first, second, and third aliquots the level of fluorescence of the dye in the plurality of cells to which the second antibody binds, wherein a decreased level of fluorescence of the dye in the second aliquot compared with the level of fluorescence of the dye in the first aliquot indicates efflux of the dye from the tumor cell, and wherein an increase in the level of fluorescence of the dye in the third aliquot compared with the level of fluorescence of the dye in the second aliquot indicates that the agent inhibits efflux of the dye from the tumor cell, thereby indicating that the tumor cell has multi-drug resistance activity.

4. The method of any one of claims 1-3 wherein the biological sample is selected from blood, bone marrow, lymph node, cerebrospinal fluid, ascites fluid, pleural fluid, pericardial fluid, peritoneal fluid, and lavage fluid.

5. The method of any one of claims 1-3 wherein the biological sample is bone marrow.

6. The method of any one of claims 1-3 wherein the biological sample is blood.

7. The method of any one of claims 1-3 wherein the biological sample is obtained from a subject who has a malignant condition.

8. The method of any one of claims 1-3 wherein the plurality of cells comprise a heterogeneous mixture of cell types.

9. The method of any one of claims 1-3 wherein the normal cell is a natural killer cell.

10. The method of any one of claims 1-3 wherein the first cell surface antigen is CD11b and wherein the normal cell is a natural killer cell.

11. The method of any one of claims 1-3 wherein the second cell surface antigen is selected from CD45, CD34, CD33, CD13, and CD38.

12. The method of any one of claims 1-3 wherein the tumor cell is a leukemic blast cell.

13. The method of any one of claims 1-3 wherein the second cell surface antigen is CD45, and wherein the tumor cell is a leukemic blast cell.

14. The method of any one of claims 1-3 wherein the second cell surface antigen is CD34, and wherein the tumor cell is a leukemic blast cell.

15. The method of any one of claims 1-3, wherein the method further comprises contacting the biological sample and the dye with a third antibody that specifically binds to a third cell surface antigen that is present on a tumor cell, and wherein the third antibody is detectably labeled with a third fluorophore that has an emission spectra distinguishable from the emission spectra of the first and second fluorophores and the dye.

16. The method of claim 15 wherein the second antibody specifically binds to cell surface antigen CD45 and the third antibody binds to cell surface antigen CD34, and wherein the tumor cell is a leukemic blast cell.

17. The method of any one of claims 1-3 wherein the method is a cytofluorimetric method.

18. The method of claim 17 wherein the cytofluorimetric method is a flow cytofluorimetric method.

19. The method of claim 17 wherein the cytofluorimetric method is an immunocytofluorimetric method.

20. The method of claim 3 wherein the malignant condition is a leukemia.

21. The method of any one of claims 1-3 wherein the fluorescent dye is selected from DuIC, DiOC, Rhodamine 123, and JC-1.

22. The method of any one of claims 1-3 wherein the fluorescent dye is DiIC.

23. The method of any one of claims 1-3 wherein the first antibody is directly or indirectly labeled with the first fluorophore.

24. The method of any one of claims 1-3 wherein the second antibody is directly or indirectly labeled with the second fluorophore.

25. The method of claim 15 wherein the third antibody is directly or indirectly labeled with the third fluorophore.

26. The method of any one of claims 1-3 wherein the first antibody is a monoclonal antibody, or an antigen-binding fragment thereof.

27. The method of any one of claims 1-3 wherein the second antibody is a monoclonal antibody, or an antigen-binding fragment thereof.

28. The method of claim 15 wherein the third antibody is a monoclonal antibody, or an antigen-binding fragment thereof.

29. The method of any one of claims 1-3 wherein the tumor cell is a leukemia cell or a lymphoma cell.

30. The method of claim 29 wherein the leukemia cell is selected from an acute myelogenous leukemia cell, a chronic myelogenous leukemia cell, an acute lymphocytic leukemia cell, and a chronic lymphocytic leukemia cell.

31. The method of claim 29 wherein the lymphoma cell is selected from the group consisting of a Hodgkin's lymphoma cell, a non-Hodgkin's lymphoma cell, a T lymphoblastoid lymphoma cell, and a B lymphoblastoid lymphoma cell.

32. The method of any one of claims 1-3 wherein detection of any one of (a) the first antibody specifically binding to the first cell surface antigen and (b) the second antibody specifically binding to the second cell surface antigen, comprises detection of a binding event between an avidin molecule and a biotin molecule.

33. The method of claim 15 wherein detection of detection of the third antibody specifically binding to the third cell surface antigen comprises detection of a binding event between an avidin molecule and a biotin molecule.
